(19) United States
(12) Patent Application Publication
(10) Pub. No.: US 2008/0221681 A1
(43) Pub. Date: Sep. 11, 2008

(54) Methods for Improving Fatigue Performance of Implants With Osteointegrating Coatings

(75) Inventors: Hai H. Trieu, Cordova, TN (US); Frank Bono, Collierville, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/684,467

(22) Filed: Mar. 9, 2007

(51) Int. Cl. A61F 2/02

(52) U.S. Cl. 623/11.11; 29/592

(57) ABSTRACT

A method which may be used for introducing a residual compressive stress into a body portion of an implantable device configured for implantation in a patient. The body portion may include an outer surface. The method also may include texturing the outer surface of the implantable device to increase a roughness of the outer surface. The outer surface may be coated with an osteointegrating material to increase osteointegration.

### FIELD OF THE INVENTION

The present invention relates generally to the field of preparing medical devices for implantation.

### BACKGROUND

Prosthetic implants are commonly used to reinforce or replace bone structure. Some of these include a coating that interfaces with the bone structure. Surface texturing may improve the adhesion of the coating onto an implant. While potentially increasing the adhesion between the coating and the implant, some surface roughening procedures also may decrease the implant's resistance to fatigue failure. Increasing resistance to fatigue in an implant with an outer coating may extend the recommended life cycle of the implant.

The present disclosure is directed to a method of maintaining the fatigue performance of an implant, where that implant has a surface coated with a material that aids in osteointegration.

### SUMMARY

In one exemplary aspect, this disclosure is directed to a method comprising introducing a residual compressive stress into a body portion of an implantable device configured for implantation in a body. The body portion may include an outer surface. The method also may include texturing the outer surface of the implantable device to increase a roughness of the outer surface. The outer surface may be coated with an osteointegrating material to increase osteointegration.

As used herein, the terms “osteointegrating material” and “osteointegrating coating” are meant to include osteoconductive coatings, osteoinductive coatings, other coatings, and any mixture, laminate, or combination thereof.

In one aspect, coating the outer surface with an osteointegrating material may include applying a coating of an osteoconductive material on the outer surface. In another aspect, coating the outer surface with an osteointegrating material may include applying a coating of an osteoinductive material on the outer surface. In yet another aspect, coating the outer surface with an osteointegrating material may include applying a coating of a mixture of an osteinductive and osteoconductive coating on the outer surface.

In another exemplary aspect, this disclosure is directed to a method of treating an implantable device including a body portion with an outer surface to maintain fatigue resistance properties. The method may include peening the outer surface of the body portion of the implantable device to introduce a residual compressive stress into the body portion, the residual compressive stress having a first depth. The method also may include texturing the peened outer surface of the body portion to increase a surface roughness of the outer surface, the texturing having a second depth into the body portion. The outer surface may be coated with an osteointegrating material to promote osteointegration.

In yet another exemplary aspect, this disclosure is directed to an implantable device that may include a body portion having an outer surface and a thickness. The body portion may include a residual compressive stress extending to a first depth. The outer surface also may include a roughened texture penetrating the outer surface of the body portion to a second depth. The second depth may be less than the first depth. An osteointegrating coating may be disposed on the outer surface and may be engaged with the roughened texture.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is an illustration of an exemplary embodiment of a vertebral member having two bone-engaging implants.

FIGS. 2A-2D are illustrations of a cross sectional view of an exemplary portion of the implant illustrated in FIG. 1. FIG. 2D also includes a stress graph illustrating stress in the exemplary portion of the implant.

FIGS. 3A-3C are illustrations of a cross sectional view of the exemplary portion of the implant illustrated in FIGS. 2A-2D. FIG. 3C also includes a stress graph illustrating stress in the exemplary portion of the implant.

FIGS. 4A-4C are illustrations of a cross sectional view of the exemplary portion of the outer surface illustrated in FIG. 3C.

FIG. 5 is an illustration of a cross section of an exemplary portion of the outer surface according to one embodiment.

FIG. 6 is an illustration of a cross section of an exemplary portion of the outer surface according to one embodiment.

FIGS. 7-10 are illustrations of exemplary implants having only a portion of the outer surface treated with the osteointegrating material.

FIGS. 11-16 are illustrations of exemplary embodiments of implantable devices treated according to the process disclosed herein.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The systems, devices, and methods described herein may be used to increase the projected useful life of bone-engaging implants. Some conventional implants include outer surfaces which have been treated to introduce surface irregularities, or treated to increase surface roughness. Increased surface roughness may cooperate with applied osteointegrating coatings to frictionally secure the coatings in place. For example, the roughening, or texturing, may increase the overall surface area available for interfacing with coating and irregularities and imperfections may receive the coating, thereby provide a physical barrier to coating displacement.

A roughened surface, although advantageous for securing a coating on an implant, may be detrimental to an implant's fatigue strength. Forces or loads repeatedly introduced during shifting of weight, such as during patient movement, may impart cyclic stress on the implant. This cyclic stress presented over time may fatigue the implant, lowering its estimated useful life.

Stress typically concentrates at certain physical features, such as at sharp corners and at cracks in the surface. A microscopic, or near microscopic crack in the surface of a load bearing object may present a potential stress concentration as a stress riser. Cyclic loading of a surface impaired with such a stress riser may lead to a shortening of the product's useful life.

For example, in a conventional material, a tensile stress concentrated at a crack tends to pull the crack open. As the crack opens, the root of the crack travels deeper into the surface, thereby further reducing a cross-section of the material available to resist the tensile load. Thus, even more stress is concentrated into the crack. Each time the tensile load is applied, the crack deepens, and if unabated, the repetitive, or cyclic loading may drive the crack deeper until the load bearing object is rendered unusable by such fatigue cracking.

Increasing resistance to effects of cyclic loading, for example, as against tensile forces acting on the surface, may increase an implant's projected life. More particularly, using the systems, devices, and methods disclosed herein, increased resistance to fatigue failure may be achieved while still maintaining the implant's ability to cooperate with the bone to frictionally or biomechanically engage the bone.

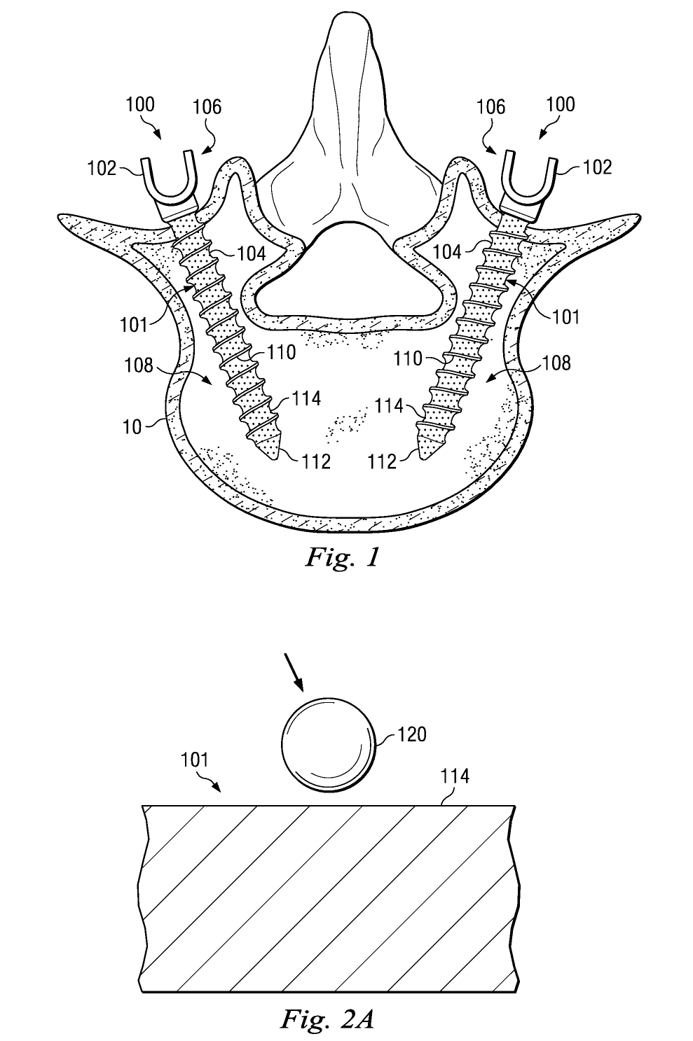

Turning now to FIG. 1, an exemplary embodiment of a vertebral member 10 is illustrated having two bone-engaging implants, each generally referenced by the numeral 100, mounted within. In this embodiment, each of the implants 100 is substantially the same although it is understood that embodiments with multiple implants 100 may include different features or specifications. Each implant 100 includes a body 101 and a coating applied to the body 101. Here, the body 101 includes a head 102 and a shaft 104. The head 102 is disposed at a proximal end 106 of the implant 100 and the shaft 104 forms a distal end 108. The shaft 104 includes radially extending threads 110 spiraling from the head 102 to a tip 112 at a distal most end. In this exemplary embodiment, the shaft 104 of the body 101 includes an outer surface 114 coated with an osteointegrating coating configured to interface directly with the bone tissue of the vertebral member 10.

In order to increase the estimated useful life of the implant, the outer surface 114 (or a portion thereof) of the body 101 may be treated to maintain fatigue resistance properties. In this example, the outer surface 114 may be treated to first introduce compressive stress to the implantable device, which may be followed by a treatment to roughen or texture the outer surface of the implant, which may be followed by a treatment to apply an osteointegrating coating to the textured surface.

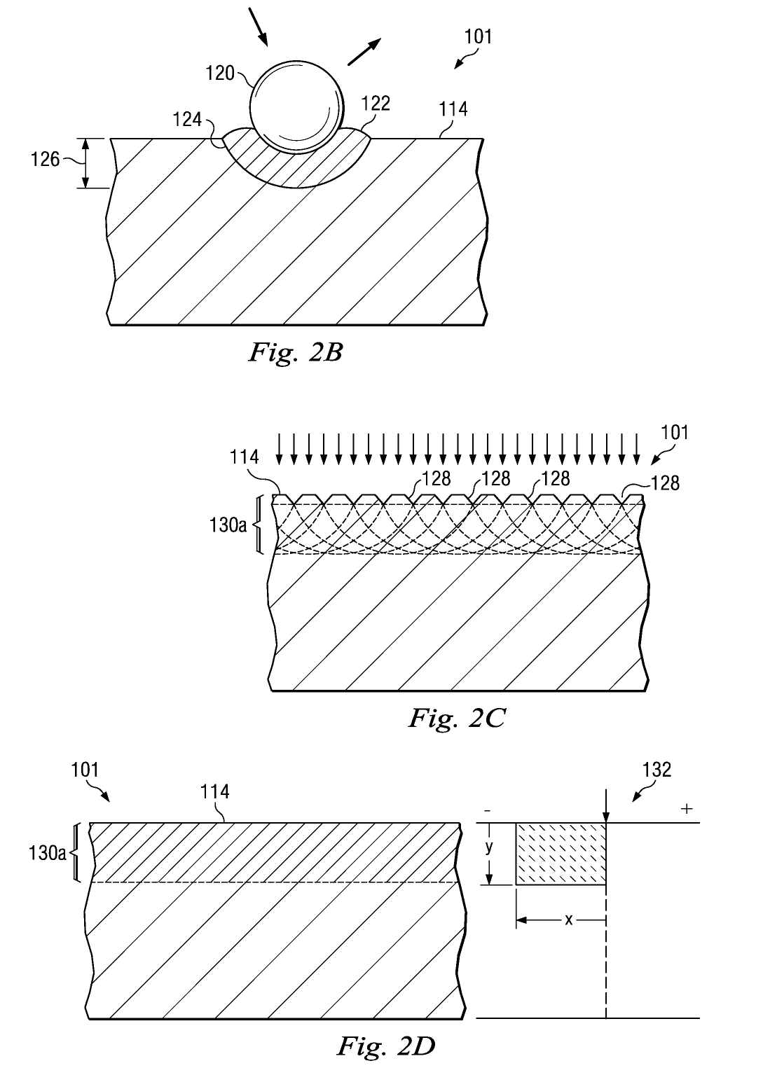

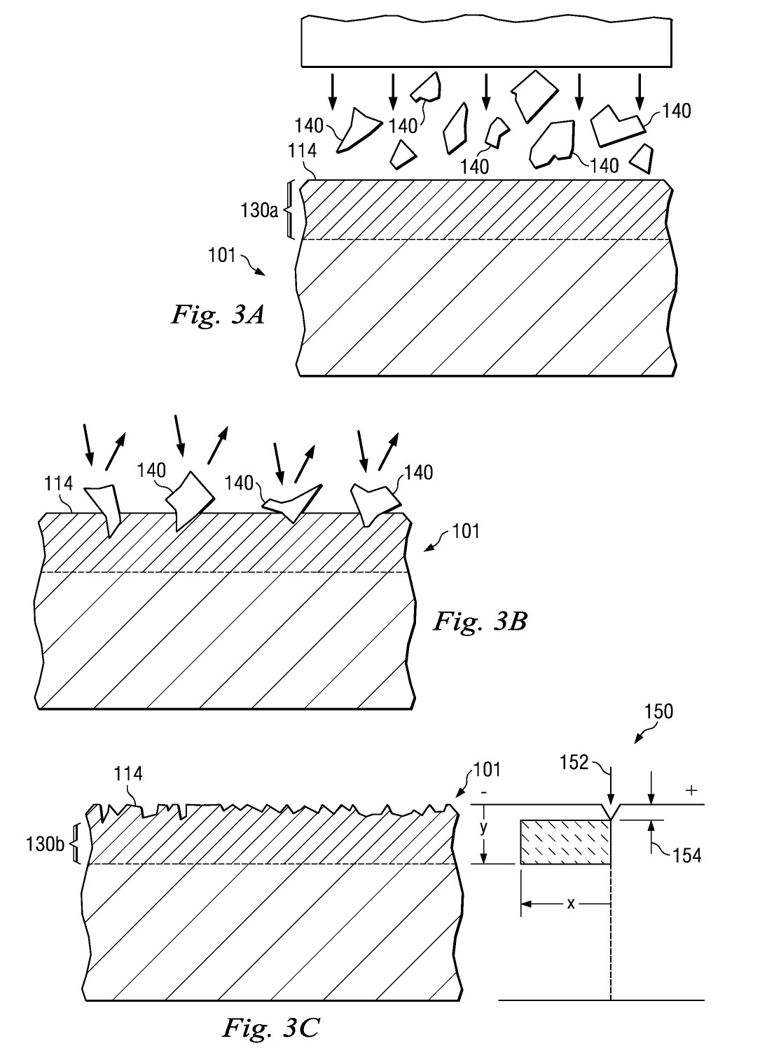

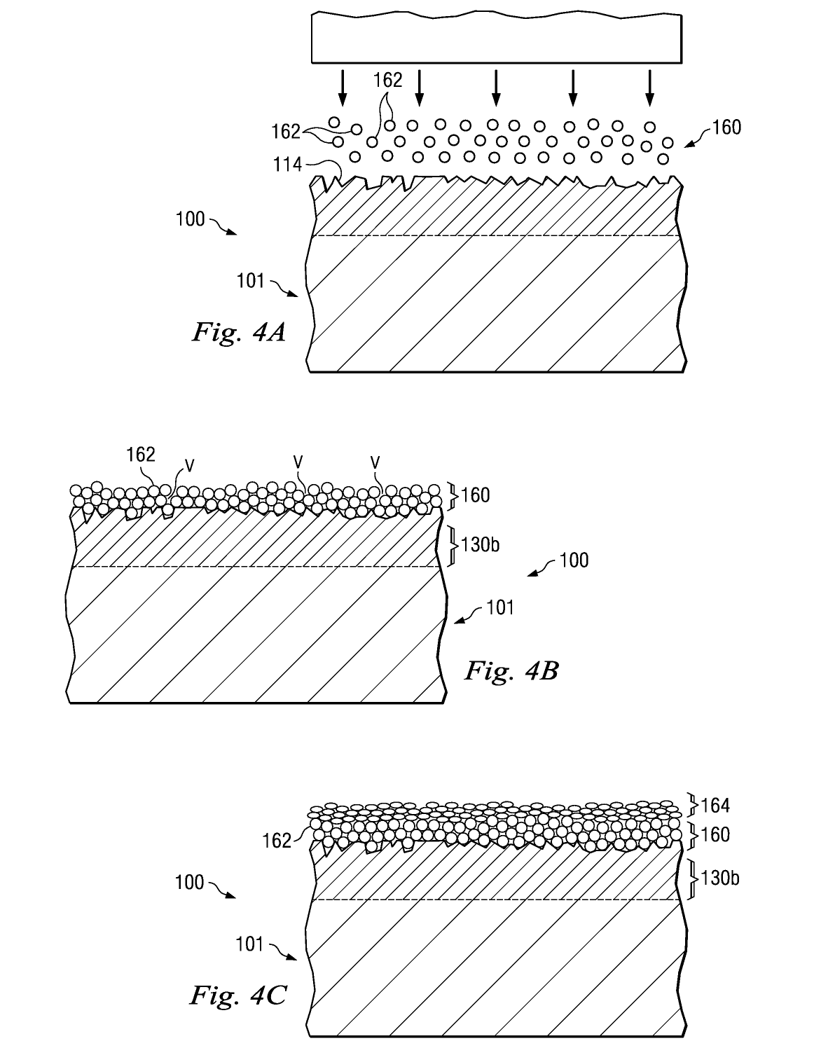

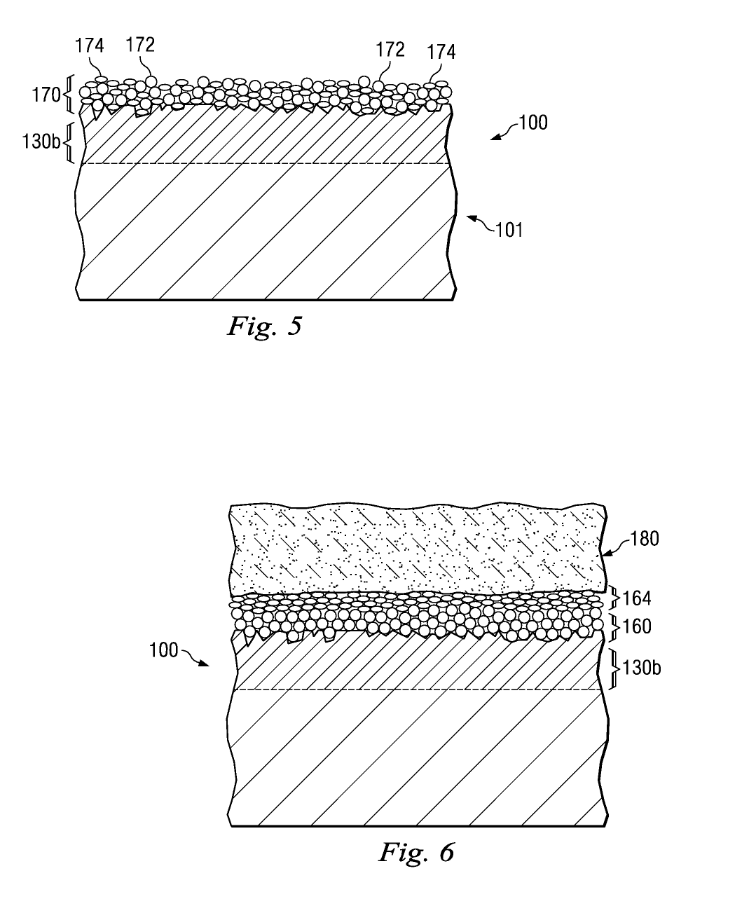

One exemplary process for treating the outer surface 114 is described with reference to FIGS. 2A-2D, FIGS. 3A-3C and FIGS. 4A-5. In this example, the outer surface 114 of the implant body 101 is work-hardened and in this case, cold-hardened, by shot peening the outer surface 114. The process for doing this, including its effects, are described below with reference to FIGS. 2A-2D. Following the work-hardening, an exemplary surface texturing process, grit blasting in this example, is described with reference to FIGS. 3A-3C. And following the texturing process, an exemplary surface coating process is described with reference to FIGS. 4A-4C and 5.

The body and surface of the implant 100 may be described as being comprised of many layers of atoms arranged in a lattice, or matrix. Spaces or voids, as well as out-of-place metal atoms or interstitials, are interspersed throughout the matrix. It is possible to force interstitials, along with otherwise aligned atoms, into these voids in a deeper surface layer. Plastic deformation occurs during the permanent dislocation of a metal atom, resulting in a breaking of existing atomic bonds followed by subsequent re-bonding in a new location. If a dent is limited to a dimple on only one side of a work piece, such as an implant, the atoms have been compressed into a smaller space. The area of the plastic deformation contains more atoms, hence more electrical bonds. As more and more atoms occupy the same space in a metal, that space's ability to deform plastically diminishes so that working more metal atoms in the same space creates a stronger metal, albeit with less ductility.

Thus, the compression of metal atoms en mass both hardens and strengthens the material. This strengthening of the metal is termed strain-hardening, or work-hardening, and it is accomplished through plastic deformation. In addition, work-hardening results in a residual compressive stress. Essentially, any applied tensile forces may be countered by the compressive force already existing in the surface layers. Shot peening is one method of work-hardening a material, such as the body 101 and outer surface 114 of the implant 100.

Referring now to FIGS. 2A-2D, FIG. 2A illustrates a cross section of an exemplary portion of the body 101 of the implant 100, with the outer surface 114. A single shot 120 is represented as a spherical or round ball and is illustrated traveling towards the outer surface 114. At impact, as illustrated in FIG. 2B, a large amount of kinetic energy is transferred from the single shot 120 to the body 101 and the outer surface 114. However, the single shot 120 maintains a portion of its kinetic energy enabling it to rebound away from the outer surface 114.

Although some energy is dissipated as heat and other energy potentially lost through break-up of a shot particle, the remaining energy is transferred into the body 101 and outer surface 114. The instantaneous transference of energy upon impact physically displaces a volume of metal at the point of impact, as illustrated in FIG. 2B. An impact of sufficient intensity plastically deforms the displaced metal, leaving a dimple after the single shot 120 travels away. A lesser amount of energy might only elastically deform the displaced metal, thereby leaving a surface mechanically unaffected.

Upon impact, an unconstrained portion of the displaced metal plastically deforms into free space on either side of the single shot 120, forming ridges 122 with a raised, rounded edge.

A constrained portion 124 of the area around the impact is bounded and unable to plastically deform into free space. The constrained portion 124 is work-hardened as a certain volume of metal atoms is compressed into a lesser volume. The work-hardened or constrained portion 124 has a thickness or depth 126 in the body 101 that corresponds to the amount of energy imparted upon impact of the single shot 120.

FIG. 2C illustrates the results of further peening of the outer surface 114. Dimples 128 created by peening begin to overlap, resulting in a uniform compressive layer 130a in the body 101. The compressive layer 130a squeezes the grain boundaries of the outer surface material together, creating a layer of crack-resistant material. Thus, the ability of the implant to resist fatigue cracking is increased.

FIG. 2D illustrates the outer surface 114 having the compressive layer described in FIG. 2C after additional peening, where the high points are eventually compacted down leaving a dimpled surface (not shown). In addition, FIG. 2D illustrates a stress graph 132 representing the corresponding stresses and their magnitudes of the implant 101 in FIG. 2D. The stress graph 132 is a simplified graphical representation of the stress experienced at a point as the stress travels down through the outer surface 114. On the stress graph 132, the negative symbol represents compressive stress while the positive symbol represents tensile stress. A residual compressive stress, due to the peening, is illustrated by this stress graph 132. The horizontal distance x away from the vertical axis represents the relative magnitude of the compressive stress at vertical depth y. The stress graph 132 is bounded horizontally by an upper surface and a lower surface of the implant. Beyond vertical depth y, the residual compressive stress is nominal. The vertical depth y indicates how deep the compressive stress extends into the outer surface 114.

The depth of the compressive layer in shot peening is dependent on a number of controllable factors, including shot size, shot material, shot velocity, distance between the surface and the nozzle, angle of impact and time under shot peen. Other considerations include the repair status of the shot peen device, the degradation of the shot peen media over time and the internal degradation of the shot peen device over time.

While shot peening in general can change the appearance of a surface, only the deeper, plastically-deforming dimples result in improved mechanical properties. Therefore, it is useful to be able to determine the depth and consistency of coverage.

Generally, there are two measurements used to verify the shot peening process. “Coverage” refers to the degree of overlap of dimples that is attained. Coverage can be examined visually and directly. “Intensity” refers indirectly to the amount of plastic deformation imparted to the target material.

However, the intensity and consistency of coverage cannot be directly equated to desired mechanical conditions without resorting to destructive test methods. Non-destructive test methods such as X-ray radiography, mag-particle inspection, ultrasonic testing, visual inspection, dye penetrant inspection, eddy current testing, and coupon testing and correlation, among others, can be used as indirect measures of depth and consistency.

One method of verifying coverage and intensity employs Almen strips. These uniform steel test coupons physically deform under peening, indicating the coverage and intensity. These may be used in test experiments that subject the same implantable device to increasing amounts of peening time. Other factors may be held constant throughout the experiment such as shot velocity, location of the implant, shot size and quality, angle of impact, material and shape of the implant and Almen strip manufacturing lot, among others. In conjunction with the shot peening of the sample implant, an Almen strip may be shot peened under the same controlled conditions. The peening time may then be increased, and the test is repeated. As residual compressive stresses accumulate, the Almen strip test coupon begins to curve. At each setting, the curvature of the Almen strip may be measured, and a corresponding implant may be destructively tested by a metallographic sampling process, among other processes. When the metallurgical sample exhibits the desired depth and consistency of shot peening, the curvature of the corresponding Almen strip will be measured and charted. The correlation between Almen strip curvature and actual surface compression produces a reliable and repeatable verification method. Hence, the shot peen process can then be manipulated as desired while ensuring that the process imparts a consistent depth of compression to the shot-peened surface.

Various implant features and base materials require varying process controls to obtain a sufficient compressive depth. One variable is the type and geometry of the shot media, which should be selected so as to not have an adverse effect on the target material's metallurgy or surface strength. The media, or shot may be made from cast steel, conditioned cut wire steel, glass, and ceramic, among other materials. The shape of shot may be approximately round as in the case of conditioned cut wire, or actually spherical as in the case of ball bearings.

One experienced in the art of shot peening will be familiar with other variations in establishing the correlation data for verification of the process and the best parameters and machinery to use for a particular implant. In some applications an implant may require partial masking to protect sensitive portions.

The resulting surface after shot peening includes small rounded ridges and dimples. In order to further improve the outer surface 114 to promote additional surface bone-engaging texturing, the outer surface 114 in some embodiments is exposed to additional processing. This additional processing creates a surface that may promotes additional bone integration, frictional resistance against implant displacement or resistance to spalling of an applied coating.

A processing step following shot peening applies a further more random roughening of the surface. In this embodiment, the outer surface 114 is textured, or roughened, beyond what is attainable through shot peening alone.

FIG. 3A-3C illustrates an exemplary process for roughening or texturing the surface 114 of the implant body 101 after introducing residual compressive stress by cold-working.

Turning to FIG. 3A, the work-hardened outer surface 114, with its compressive layer 130a, is subjected to an additional texturing treatment. In this embodiment, the texturing is accomplished by grit blasting. This includes pneumatically hurling grit particles 140 at a high velocity at the outer surface 114. Unlike the shot peen media described above, the grit particles 140 contain edges, corners, and non-uniform sizes and shapes. FIG. 3B illustrates some of the grit particles 140 engaging the outer surface 114 of the implant 100. Corners and edges of the grit particles create small impressions, gouges, and the like in the outer surface 114 by plastic deformation or material removal, thereby roughening the surface. This may increase the overall surface area of the outer surface, thereby increasing the capacity of the outer surface 114 to mechanically interlock, and otherwise adhere, to an osteointegrating coating.

The grit blasting process may include any known grit, which is selected based on a survey of the target material used for an implant. Grit particles may be formed of glass, sand, metal, polymers, slag, alumina oxide, among others. Typically, though not always, the selected grit particles are harder than the implant material.

Grit blasting alone, while useful for improving coating adhesion, can create stress risers leading to a shortened useful life. FIG. 3C illustrates the outer surface 114 after the texturing process in conjunction with a corresponding stress graph 150. As can be seen, the outer surface 114 includes irregularities such as notches and nicks that increase the surface roughness of the implant 100. These irregularities reduce some of the residual compressive stress introduced during the shot peening process; however, the irregularities do not fully penetrate the compressive layer 130a. This phenomenon is further illustrated by the stress graph 150. In this simplified graphical representation of the stress experienced in the outer surface 114, an exemplary notch 152, representing a notch in the outer surface 114, is illustrated in the stress graph 150. The notch 152 represents a removed portion of the outer surface 114, resulting in a decrease of the residual compressive stress down to a notch depth 154. Hence, the total compressive stress illustrated in the stress graph 150 of FIG. 2C has been reduced by the difference between the notch depth 154 and the vertical distance y illustrated in FIG. 3C. In effect, a tensile stress at the outer surface 114 is met with less counter-acting compressive residual stress. However, as can be seen, a relative amount of residual compressive strength 130b remains beyond the notch 152, providing resistance to crack propagation. This benefit may continue to inure as long as the notch depth 154 is less than the vertical depth y of the residual compressive stress. In some exemplary embodiments, the roughening process is established to roughen the outer surface 114 to a depth that is less than about 50% of the depth of the compressive layer. Other depths, both greater and smaller also are contemplated.

Thus, as illustrated in FIG. 3C, by carefully controlling the shot peening and grit blasting processes, a residual compressive stress benefit can be combined with a surface roughening benefit.

The roughened outer surface 114 of FIG. 3C is further enhanced to promote osteointegration by the addition of an osteointegrating coating. The osteointegrating coating may comprise, for example, a combination of osteoconductive and osteoinductive materials. An exemplary process for applying the osteointegrating coating is described with reference to FIGS. 4A-4C.

Turning to FIG. 4A, in one example, a thermal spray process is used to deposit an osteoconductive coating 160 onto the outer surface 114. Some of the osteoconductive particles 162 in the coating 160 may be melted by the thermal spray process and forced into the cracks, scores, and markings on the outer surface 114, thereby securing the coating 160 to outer surface 114 of the implant body 101.

As illustrated in FIG. 4B, the exemplary osteoconductive coating 160, though mechanically and frictionally bonded to the outer surface 114, may not be completely solid. Pores, or voids V, in the osteoconductive coating 160 may promote bone formation and ingrowth. In this embodiment, an osteoconductive coating of hydroxyapatite (HA) is applied using a plasma deposition process. The osteoconductive coating may provide a favorable scaffolding for vascular ingress, cellular infiltration and attachment, cartilage formation, calcified tissue deposition, or any combination thereof. The osteoconductive coating may be used alone or in conjunction with an osteoinductive material.

Turning to FIG. 4C, the osteoconductive coating 160 has been further enhanced with an osteoinductive coating 164. In this example, the osteoinductive coating attaches to the pores and voids in the osteoconductive coating 160. The osteoinductive coating may reside on, below, and/or in the osteoconductive coating. In one embodiment, the osteoinductive coating 164 may be applied by soaking the outer surface 114 in a solution containing an osteoinductive material, such as for example, bone morphogenetic protein (BMP). The solution may penetrate the osteoconductive coating or may reside on top of the osteoconductive coating. The outer surface 114 may then be dried, leaving a layer of the osteoinductive material above and/or among, the particles of the osteoconductive coating.

In addition to thermal spraying, plasma deposition and immersion in a solution, an osteointegrating coating may be applied by a process such as, for example, vapor deposition, electroplating, dip-coating, or non-thermal spraying.

FIG. 5 illustrates one example of a single osteointegrating coating 170 applied to the outer surface 114. Here, the single osteointegrating coating 170 contains both osteoconductive and osteoinductive material 172, 174.

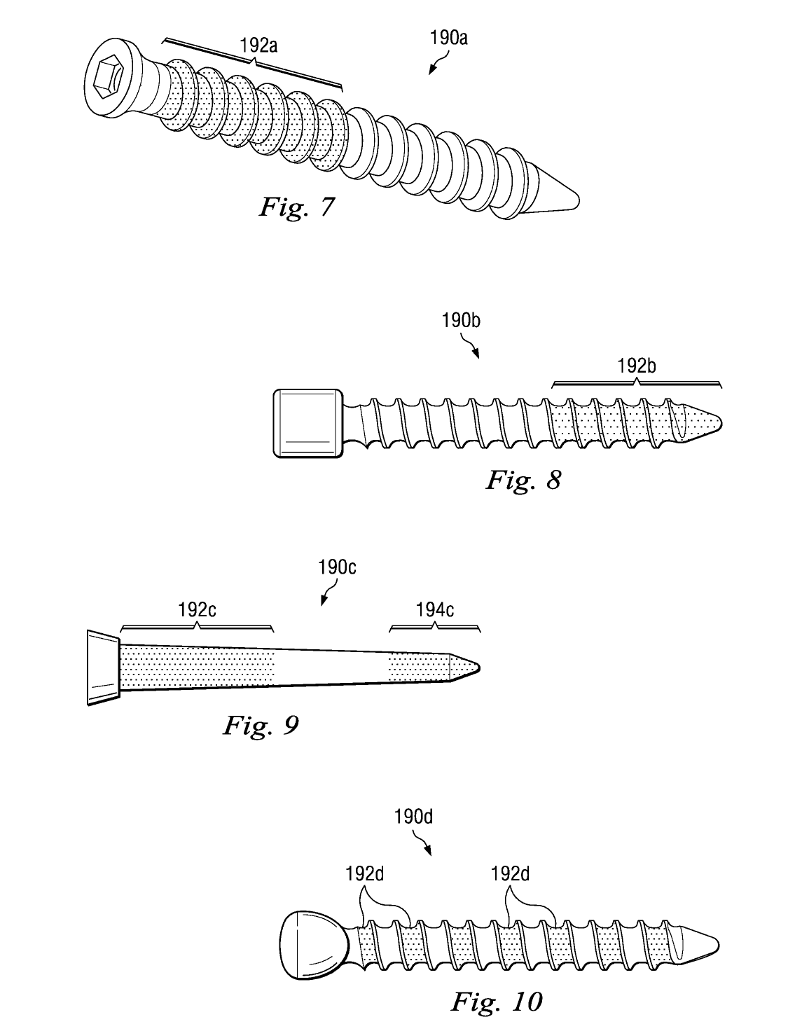

FIG. 6 illustrates one example of the implant 100 in a desired position near bone 180. Bone 180 directly interfaces with the dual osteointegrating coatings of osteoconductive and osteoinductive materials 160, 164. Over time, boney tissue grows throughout the osteointegrating coatings 160, 164, thereby mechanically securing the bone 180 to the implant 100, and thereby fixing the position of the implant 100. In addition, the residual compressive layer 130b continues to inhibit crack propagation at the outer surface, thereby prolonging the estimated useful life of the implant 100.

Now returning to FIG. 1, the shaft 104 of the body 101 of the implant 100, including the outer surface 114 is work-hardened, textured, and then coated with an osteointegrating material to improve bony apposition. However, in other exemplary embodiments, only a portion of the outer surface 114 of the body 101 is work-hardened, textured, and coated. For example, in some exemplary embodiments, the entire implant body 101 is work-hardened, but only the outer surface 114 is textured and coated. In other exemplary embodiments, only a part of the outer surface 114 is work-hardened, textured, and coated. In yet other exemplary embodiments, only a portion of a textured area is coated. Other combinations also are contemplated.

The osteointegrating material may improve the connection between the implant and outer, cortical bone tissue of vertebral member 10. In some exemplary embodiments, the osteointegrating material may be positioned to contact the cancellous bone tissues of vertebral member 10. In addition to coating the outer surface 114 with osteointegrating material, the osteointegrating material may be partially or wholly impregnated into the implant body 101.

The coating may be applied to a textured surface at any suitable time period and in any suitable manner. For example, in one embodiment, the osteointegrating coating is applied during the time of the surgical procedure. This may be achieved by using a paste. In other embodiments, the osteointegrating coating is applied as a manufacturing step prior to shipping the implant from a manufacturing facility. Other coating times also may be used, such as during preparation for surgery.

In some embodiments, the osteointegrating coating may include two or more different osteointegrating materials. The different osteointegrating materials may be positioned along the same section of the outer surface thereby overlapping, or they may be separated, such as adjacent each other or spaced apart from each other.

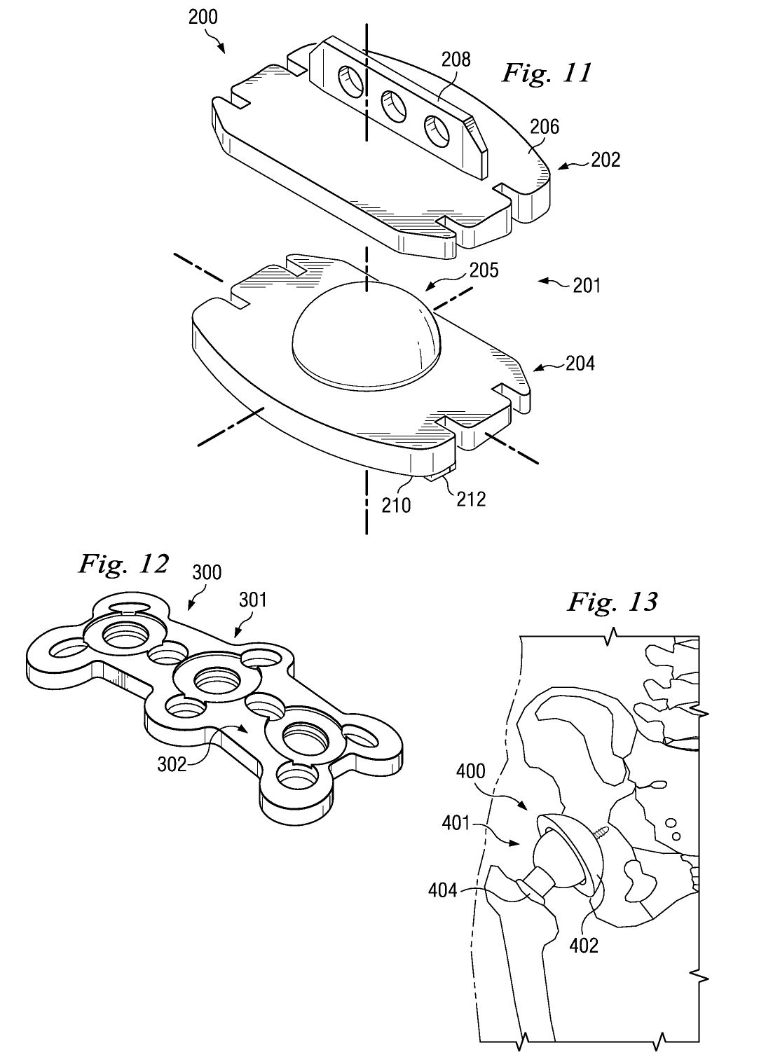

The osteointegrating coating, whether it includes only an osteoconductive coating, only an osteoinductive coating, some other osteointegrating coating, or a mixture or laminate of different coatings, may be applied to the outer surface of the implant body to cover the entire outer surface, or only a part of the entire outer surface. For example, in some embodiments, the coating may be applied only in bone-engaging portions of the outer surface. In some embodiments, the coating may be applied in certain sections of the bone-engaging surface, and not in other regions of the bone engaging surface. The coating may be applied in a pattern, in random patches, or otherwise. FIGS. 7-12 show some examples of bone screws of anchors having a coating disposed in sections, such as in patterns on the implant.

Turning to FIGS. 7-10, the osteointegrating material may be applied over the entirety of the outer surface of the body or over just a portion of the body. In these figures, a number of different types of implants 190, shown as various bone fasteners, each include an osteointegrating coating section 192. The lengths and positioning of the coating section 192 along the surfaces of the implants may vary. FIG. 7 illustrates an embodiment of an implant 190a with the coated section 192a extending along a proximal end, approximately half-way along the shaft. The coated section could extend either further or less than that shown. FIG. 8 shows an implant 190b with a coating section 192b at the distal end of the implant.

In some embodiments, two or more different osteointegrating materials are attached to implant. The different osteointegrating materials may be positioned along the same section of the implant, or may be separated. FIG. 9 illustrates an embodiment of an implant 190c with a first osteointegrating section 192c separated from a second osteointegrating section 194c. The amount of separation may vary, and as stated above, the material also may vary.

In some embodiments, such as the exemplary implant 190d shown in FIG. 10, the osteointegrating sections 192d may be interspersed along the length of the shaft. Here, the implant 190d includes a helical osteointegrating sections 192d spaced along the shaft 22, so that the coating is applied along between adjacent threads.

In yet another example, the osteointegrating coating may be applied in small patches over a surface. In yet other embodiments, a coated surface communicates with either cortical bone or cancellous bone but not both.

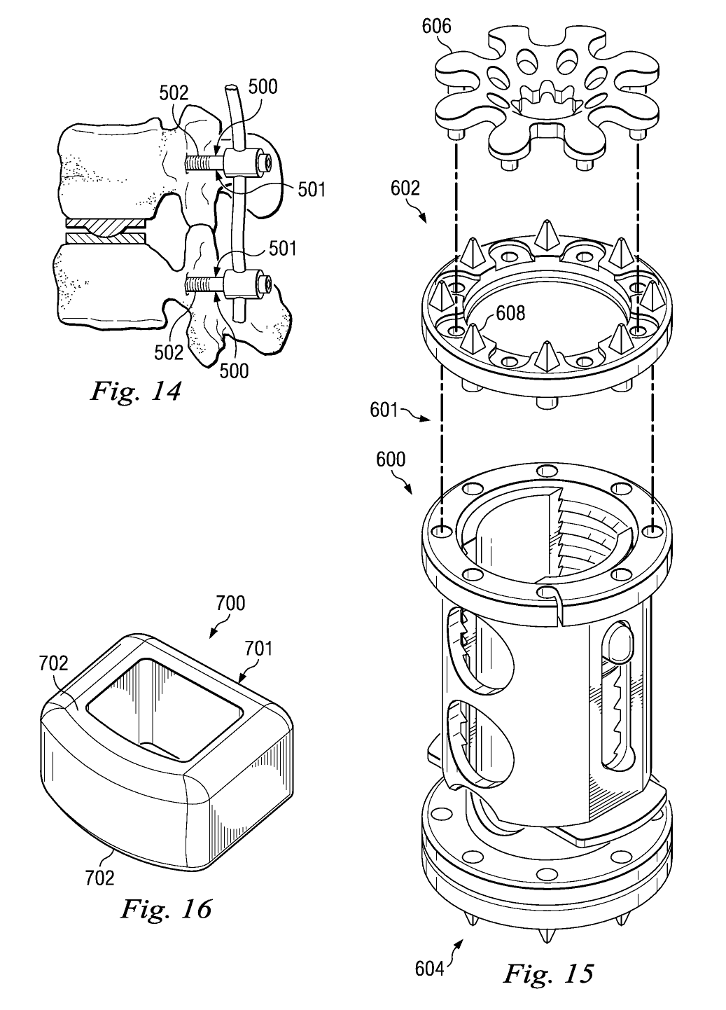

FIGS. 11-16 illustrate some examples of additional implants that may be treated to increase their life expectancy. Referring first to FIG. 11, an exemplary implant, referenced herein by the reference numeral 200 is a motion preserving spinal disc configured for implantation between adjacent vertebrae to replace a natural spinal disc. The implant 200 includes a body 201 and an osteointegrating coating on the body 201. The body 201 may be formed of an upper portion 202 and a lower portion 204 that together have features that form a ball and socket type articulating joint 205 that provides relative rotation between the adjacent vertebrae.

The upper portion 202 includes an upper surface 206 and a keel 208, while the lower surface includes a lower surface 210 and a keel 212. These surfaces 206, 210, along with surfaces of the keels 208, 212 are coated with the osteointegrating coating which interfaces with the bone tissue of the adjacent vertebrae. The outer surfaces may be treated by a work-hardening process to increase fatigue resistance, and then by a texturing process to increase the capacity of the outer surfaces to mechanically engage adjacent bone tissue, followed by application of the osteointegrating coating designed to promote boney ingrowth. In some embodiments, only the upper and lower surfaces 206, 210 are treated, while in other embodiments only the keels 208, 212 are treated. In yet other embodiments, the keels 208, 212 and the outer surfaces 206, 210 are treated. Some embodiments may include only a portion of a surface to be treated with one or both of the blasting, texturing and coating processes.

In some embodiments, the ball and socket joint components may be highly polished and any imperfections may be undesirable. Therefore, a manufacturer may desire to protect the ball and socket joint 205 from shot peening, grit blasting and coating. Accordingly, prior to processing, the ball and socket joint components may be masked so as to protect them from accidental peening, blasting, or coating.

FIG. 12 illustrates another exemplary embodiment of an implant, referenced herein by the numeral 300, that may be treated. In this exemplary embodiment, the implant is a bone plate that may span an intervertebral disc space and attach to adjacent vertebrae using implantable bone anchors. The implant includes a body 301 with outer surfaces 302 that may be resistant to fatigue and may include texturing and an osteointegrating coating. The implant may include a lower surface that may be an outer surface and may be treated to reduce fatigue and include proper surfacing.

FIG. 13 is yet another exemplary embodiment of an implant, referenced herein by the numeral 400. In this exemplary embodiment, the implant is an implantable prosthetic hip joint having a body 401, including a hip stem 404, with an outer surface 402. Any portion of the outer surface may be resistant to fatigue and may include texturing and the osteointegrating coating. The outer surface 402 and hip stem 404 may be treated through a work-hardening process and texturing and coating processes as described above to provide the desired qualities and characteristics.

FIG. 14 is yet another exemplary embodiment of an implant, referenced herein by the numeral 500. In this exemplary embodiment, the implant is, as in FIG. 1, a bone anchor. Here the bone anchor is a pedicle screw. The implant 500 includes a body 501 and a coating. A portion of the coated body protrudes into a part of the vertebra, such that the coating on the bone-engaging outer surface 502 interfaces with the vertebra. The outer surface 502 may be treated through a work-hardening process and texturing and coating processes as described above to provide the desired qualities and characteristics.

FIG. 15 is yet another exemplary embodiment of an implant, referenced herein by the numeral 600. In this exemplary embodiment, the implant 600 is a corpectomy device configured to replace a vertebral body. The implant 600 includes a body 601 that may be coated with an osteointegrating coating. Here, the body 601 includes ends 602, 604 that include bone-engaging features, such as a basket 606, spikes 608, and other surfaces. All or a part of one or more of these features and surfaces may be treated through a work-hardening process followed by texturing and coating processes as described above to provide the desired qualities and characteristics.

FIG. 16 is yet another exemplary embodiment of an implant, referenced herein by the numeral 700. In this exemplary embodiment, the implant is an intervertebral spacer configured to fit within an intervertebral space between adjacent vertebrae. The spacer includes a body 701 having outer surfaces 702 that may interface with the upper or lower vertebra. All or a part of one or more of these surfaces may be treated through a work-hardening process followed by texturing and coating processes as described above to provide the desired qualities and characteristics.

FIGS. 7-12 show a few examples of implants finding utility for the process of maintaining fatigue performance described herein. Yet other implants may be treated by the disclosed processes and include the disclosed features. Some examples of other suitable implants include a disc replacement device, a facet joint replacement implant, an interspinous spacer, a bone screw, a bone anchor, a bone fastener, a fenestrated screw, a corpectomy device, an intramedulary rod, a hip joint replacement implant, a bone pin or rod, a knee joint replacement implant, a shoulder joint replacement implant, an elbow joint replacement implant, a wrist joint replacement implant, an ankle joint replacement implant, a finger joint replacement implant, a toe joint replacement implant, a dental implant, and a maxillofacial/cranial implant. These are just example, and others also are contemplated.

The implants need not be under cyclic load to benefit from the process disclosed herein. Accordingly, any outer surface may be benefited from the processes disclosed herein. For example, in addition to the implants illustrated, the process may be used to increase the fatigue resistance and the bone-engaging properties of implantable devices, such as bone pins and bone screws. As described above, these processes may find particular utility when used on spinal implants that may be subject to cyclic loading.

Although the above example uses shot peening for the work-hardening process, other work-hardening processes also may impart a suitable compressive layer to the implant. For example, in some exemplary processes, the compressive stress layer is introduced to the implant using a forging process, a pressurization process, a water jet process, a drawing process among other processes and treatments. Cold-working treatments may be used to work harden the implant 100. Some of these may include, for example, cold rolling, roll forming, drawing, deep drawing, pressing, bending, cold forging, cold extrusion, hammering, and shearing, among others.

Alternatively, other forms of work-hardening via peening processes other than shot peening can be used. For example, laser peening uses shock waves to induce residual compressive stress. This may be useful when a very deep, or tightly controlled compressive layer is desired. Strain peening also may be used, whereby the implant is pre-strained below its elastic limit so that the bone-engaging surface is in tension is followed by shot or laser peening the surface to create a compressive layer, and then releasing the implant to impart further compression as it returns to its original form. Dual peening may be used to introduce additional compression by shot peening a second time with a smaller-sized shot.

Also, it is noted that grit blasting is just one example of a texturing process that may be used to promote bone integration and frictional resistance to displacement. Other suitable processes include, for example, chemical or electrical etching, sanding, electrical discharge, or embedding particles within the surface, among others.

It is further disclosed that treatment of the entire bone contacting surface or a portion of the bone contacting surface may be suitable to impart the strength and surface texture desired. For example, in some exemplary embodiments, such as that illustrated in FIG. 1, only a distal end portion near a tip may be coated with an osteointegrating coating or treated with the roughening and coating process, while the entire bone-engaging shaft 104 may be treated with the work-hardening process. Yet other arrangements are contemplated. In some examples, the outer surface may be treated in a pattern or spot treated with any or all of the hardening, texturing, and coating processes to achieve desired properties and a desired interface.

In some embodiments the osteointegrating coating may be either osteoconductive or osteoinductive, or both. The osteointegrating material in the coating may be heterogeneous in some examples and homogeneous in others.

In addition to, or in place of using HA (hydroxyapatite) as an osteoconductive coating, other exemplary osteoconductive coatings may comprise one or more of: biocompatible ceramics; calcium sulfate; a calcium phosphate such as HA, corraline hydroxyapatite, biphasic calcium phosphate, tricalcium phosphate, or fluorapatite; mineralized collagen; bioactive glasses; porous metals; bone particles; and demineralized bone matrix (DBM).

An osteoinductive coating may include: other forms of bone morphogenetic proteins (BMP), such as BMP-2, BMP-4, BMP-7, rhBMP-2, or rhBMP-7; demineralized bone matrix (DBM); transforming growth factors (TGF, e.g., TGF-β); osteoblast cells; growth and differentiation factor (GDF); insulin-like growth factor 1, platelet-derived growth factor, fibroblast growth factor, or any combination thereof.

In a further example, an osteoinductive coating material may include HMG-CoA reductase inhibitors, such as a member of the statin family, such as lovastatin, simvastatin, pravastatin, fluvastatin, atorvastatin, cerivastatin, mevastatin, pharmaceutically acceptable salts esters or lactones thereof, or any combination thereof. With regard to lovastatin, the substance can be either the acid form or the lactone form or a combination of both.

In yet another example, an osteoinductive material may comprise LIM mineralized proteins (LMP), osteoinductive peptides, pharmaceutical agents such as antibiotics, pain medication, anti-inflammatory drugs, steroids, osteogenic compositions such as, therapeutic or infection resistant agent, or one or more of the previous in combination.

In some embodiments, the osteointegrating coating material may include multifunctional polymeric materials that inhibit adhesion and immune recognition between cells and tissue. These materials may include a tissue-binding component and a tissue non-binding component. Specific materials may include PEG/PLL copolymers with molecular weights greater than 300, with structures that include AB copolymers, ABA copolymers, and brush-type copolymers. U.S. Pat. Nos. 5,462,990 and 5,627,233 disclose various materials and are incorporated herein by reference.

Additionally, the osteointegrating coating may use grafted polyionic copolymers that are able to attach to biological and non-biological samples to control cell-surface, cell-cell, and tissue-surface interactions as disclosed in WO 98/47948, incorporated herein by reference. The coating may also include the application of polyionic, PEG-grafted copolymers such as disclosed in U.S. Pat. No. 6,743,521, incorporated herein by reference.

In one embodiment, the osteointegrating coating contains grafted non-interactive material such as PEG (polyethylene glycol) or PEO (polyethylene oxide) within the polymer. Another example coating may be a combination wherein the polymer is a PEG-grafted poly (amino acid) with a polycationic backbone made of lysine, histidine, arginine or ornithine in D-, L-, or DL-configuration, or the polymer is a PEG-grafted polymer with a cationic backbone of a polysaccharide such as chitosan, partially deacetylated chitin, and amine-containing derivatives of neutral polysaccharides, or the polymer is a PEG-grafted non-peptide polyamine with a polycationic backbone such as poly (aminostyrene), poly (aminoacrylate), poly (N-methyl aminoacrylate), poly (N-ethylaminoacrylate), poly (N,N-dimethyl aminoacrylate), poly (N,N-diethylaminoacrylate), poly (aminomethacrylate), poly (N-methyl amino-methacrylate), poly (N-ethyl aminomethacrylate), poly (N,N-dimethyl aminomethacrylate), poly (N,N-diethyl aminomethacrylate), poly (ethyleneimine), polymers of quaternary amines, such as poly (N,N, N-trimethylaminoacrylate chloride), poly (methacrylamidopropyltrimethyl ammonium chloride), or the polymer is a PEG-grafted charged synthetic polymer with a polycationic backbone such as polyethyleneimine, polyamino (meth) acrylate, polyaminostyrene, polyaminoethylene, poly (aminoethyl) ethylene, polyaminoethylstyrene, and N-alkyl derivatives thereof.

Other embodiments include one more coatings comprising a copolymer, wherein the copolymer is a PEG-grafted copolymer with an anionic backbone of a poly (amino acid) grafted with poly (ethylene glycol) where the amino acid contains an additional pendant carboxy group imparting a negative charge to the backbone at pH above 4 and in particular at neutral pH such as polyaspartic acid or polyglutamic acid; or a natural or unnatural polymer with pendant negatively charged groups, particularly carboxylate groups, including alginate, carrageenan, furcellaran, pectin, xanthan, hyaluronic acid, heparin, heparan sulfate, chondroitin sulfate, dermatan sulfate, dextran sulfate, poly (meth) acrylic acid, oxidized cellulose, carboxymethyl cellulose and crosmarmelose, synthetic polymers and copolymers containing pendant carboxyl groups, such as those containing maleic acid or fumaric acid in the backbone. Examples of these materials are disclosed in U.S. Pat. No. 5,567,440, herein incorporated by reference.

In yet another embodiment, the osteointegrating coating comprises nanoparticles, wherein each particle is generally less than 500 nm in diameter. The nanoparticles act to reduce protein “denaturation” as well as subsequent foreign body reactions. Nanoparticles may include a metal particle, carbon particle, inorganic chemical particle, organic chemical particle, ceramic particle, graphite particle, polymer particle, protein particle, peptide particle, DNA particle, RNA particle, bacteria/virus particle, hydrogel particle, liquid particle or porous particle. Thus, the nanoparticles may be, for example, metal, carbon, graphite, polymer, protein, peptide, DNA/RNA, microorganisms (bacteria and viruses) and polyelectrolyte. Polymers may include copolymers of water soluble polymers, including, but not limited to, dextran, derivatives of poly-methacrylamide, PEG, maleic acid, malic acid, and maleic acid anhydride and may include these polymers and a suitable coupling agent, including 1-ethyl-3(3-dimethylaminopropyl)-carbodiimide, also referred to as carbodiimide. Polymers may be degradable or nondegradable or of a polyelectrolyte material. Degradable polymer materials include poly-L-glycolic acid (PLGA), poly-DL-glycolic, poly-L-lactic acid (PLLA), PLLA-PLGA copolymers, poly(DL-lactide)-block-methoxy polyethylene glycol, polycaprolacton, poly(caprolacton)-block-methoxy polyethylene glycol (PCL-MePeg), poly(DL-lactide-co-caprolactone)-block-methoxy polyethylene glycol (PDLLACL-MePEG), some polysaccharide (e.g., hyaluronic acid, polyglycan, chitoson), proteins (e.g., fibrinogen, albumin, collagen, extracellular matrix), peptides (e.g., RGD, polyhistidine), nucleic acids (e.g., RNA, DNA, single or double stranded), viruses, bacteria, cells and cell fragments, organic or carbon-containing materials, as examples. Nondegradable materials include natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine), polyacroleine, polyglutaraldehyde), some polysaccharides (e.g., hydroxypropyl cellulose, cellulose derivatives, DEXTRAN, dextrose, sucrose, FICOLL, PERCOLL, arabinogalactan, starch), and hydrogels (e.g., polyethylene glycol, ethylene vinyl acetate, N-isopropylacrylamide, polyamine, polyethyleneimine, poly-aluminuin chloride). U.S. Patent Application Publication No. 2005/0084513 discloses various nanoparticles and is herein incorporated by reference.

The term “distal” is generally defined as in the direction of the patient, or away from a user of a device. Conversely, “proximal” generally means away from the patient, or toward the user.

It is understood that all spatial references, such as “top,” “inner,” “outer,” “bottom,” “left,” “right,” “anterior,” “posterior,” “superior,” “inferior,” “medial,” “lateral,” “upper,” and “lower” are for illustrative purposes only and can be varied within the scope of the disclosure.

While embodiments of the invention have been illustrated and described in detail in the disclosure, the disclosure is to be considered as illustrative and not restrictive in character. All changes and modifications that come within the spirit of the invention are to be considered within the scope of the disclosure.

We claim:

1. A method comprising:
introducing a residual compressive stress into a body portion of an implantable device configured for implantation in a patient, the body portion including an outer surface;
texturing the outer surface of the implantable device to increase a roughness of the outer surface; and
coating the outer surface with an osteointegrating material to increase osteointegration.

2. The method of claim 1, wherein the coating the outer surface with a osteointegrating material comprises at least one of:
applying a coating of an osteoconductive material on the outer surface; and
applying a coating of an osteoinductive material on the outer surface.

3. The method of claim 1, wherein the coating the outer surface includes coating with a single osteointegrating coating comprising a mixture of osteoconductive and osteoinductive material.

4. The method of claim 1, wherein the coating the outer surface includes one of: thermal spraying; plasma depositing; vapor depositing; electroplating; non-thermal spraying; applying a paste; dip-coating, and immersing in a solution.

5. The method of claim 1, wherein the coating the outer surface includes applying a coating of hydroxyapatite.

6. The method of claim 5, wherein the coating the outer surface further comprises:
soaking the outer surface of the implantable device in a solution containing osteointegrating material such that molecules of the solution bonds with the hydroxyapatite coating.

7. The method of claim 1, wherein coating the outer surface includes coating less than all of a bone engaging portion of the outer surface.

8. The method of claim 1, wherein the coating the outer surface includes applying osteointegrating material to a section of the outer surface.

9. The method of claim 1, wherein the coating the outer surface includes applying an osteoconductive material comprising at least one of: hydroxyapatite; a biocompatible ceramic; a calcium sulfate; a calcium phosphate; corraline hydroxyapatite; biphasic calcium phosphate; tricalcium phosphate; fluorapatite; mineralized collagen; bioactive glasses; porous metals; bone particles; demineralized bone matrix (DBM); and combinations thereof.

10. The method of claim 1, wherein the coating the outer surface includes applying an osteoinductive material comprising at least one of: bone morphogenetic proteins; demineralized bone matrix; transforming growth factors; osteoblast cells; growth and differentiation factors; insulin-like growth factor 1; platelet-derived growth factor; fibroblast growth factor; and combinations thereof.

11. The method of claim 1, wherein the introducing a residual compressive stress is performed until the compressive stress is at a first depth in the body portion, and wherein texturing the outer surface is performed until the texturing is at a second depth in the body portion, and wherein the second depth is less than the first depth.

12. The method of claim 1, wherein the introducing a residual compressive stress comprises work-hardening the body portion of the implantable device.

13. The method of claim 12, wherein the work-hardening includes one of:
a forging process; a pressurization process; a water jet process; a drawing process; cold rolling; drawing; deep drawing; pressing; bending; cold forging; cold extrusion; hammering; shearing; and peening.

14. The method of claim 1, wherein the texturing includes one of: chemical etching; electrical etching; sanding; electrical discharge; machining; grit-blasting; abrading; plasma etching; and embedding particles.

15. A method of treating an implantable device including a body portion with an outer surface to maintain fatigue resistance properties comprising:
peening the outer surface of the body portion of the implantable device to introduce a residual compressive stress into the body portion, the residual compressive stress having a first depth;
texturing the peened outer surface of the body portion to increase a surface roughness of the outer surface, the texturing having a second depth into the body portion; and
coating the outer surface of the body portion with an osteointegrating material to promote osteointegration.

16. The method of claim 15, wherein the second depth is less than the first depth.

17. The method of claim 15, wherein coating the outer surface with an osteointegrating material comprises at least one of:
applying a coating of an osteoconductive material on the outer surface; and
applying a coating of an osteoinductive material on the outer surface.

18. The method of claim 15, wherein the coating the outer surface includes coating with a single osteointegrating coating comprising a mixture of osteoconductive and osteoinductive material.

19. The method of claim 15, wherein the coating the outer surface includes one of: thermal spraying; plasma depositing; vapor depositing; electroplating; non-thermal spraying; applying a paste; dip-coating; and immersing in a solution.

20. An implantable device, comprising:
a body portion having an outer surface and a thickness, wherein the body portion has a residual compressive stress extending to a first depth,
wherein the outer surface has a roughened texture, the roughened texture penetrating the outer surface of the body portion to a second depth, the second depth being less than the first depth; and
an osteointegrating coating disposed on the outer surface and engaged with the roughened texture.

21. The implantable device of claim 20, wherein the osteointegrating coating comprises at least one of:
a coating of an osteoconductive material on the outer surface; and
a coating of an osteoinductive material on the outer surface.

22. The implantable device of claim 20, wherein the osteointegrating coating includes a mixture of osteoconductive and osteoinductive material.

23. The implantable device of claim 22, wherein the osteoinductive material is disposed within pores of the osteoconductive material.

24. The implantable device of claim 20, wherein the osteointegrating coating is disposed on a section of the outer surface.

25. The implantable device of claim 20, wherein the body portion is one of: an implantable artificial disc; a facet joint replacement implant; an interspinous spacer; an intervertebral spacer; a bone plate; a bone screw; a bone anchor; a bone fastener; a fenestrated screw; a corpectomy device; an intramedulary rod; a hip joint replacement implant; a bone pin or rod; a knee joint replacement implant; a shoulder joint replacement implant; an elbow joint replacement implant; a wrist joint replacement implant; an ankle joint replacement implant; a finger joint replacement implant; a toe joint replacement implant; a dental implant; and a maxillofacial/cranial implant.

26. The implantable device of claim 20, wherein the coating includes an osteoconductive material comprising at least one of: hydroxyapatite; a biocompatible ceramic; a calcium sulfate; a calcium phosphate; corraline hydroxyapatite; biphasic calcium phosphate; tricalcium phosphate; fluorapatite; mineralized collagen; bioactive glasses; porous metals; bone particles; demineralized bone matrix (DBM); and combinations thereof.

27. The implantable device of claim 20, wherein the coating includes an osteoinductive material comprising at least one of: bone morphogenetic proteins; demineralized bone matrix; transforming growth factors; osteoblast cells; growth and differentiation factors; insulin-like growth factor 1; platelet-derived growth factor; fibroblast growth factor; and combinations thereof.
